(19) United States
(12) Patent Application Publication
(10) Pub. No.: US 2025/0221697 A1
(43) Pub. Date: Jul. 10, 2025

(54) DEVICES, SYSTEMS, AND METHODS FOR TISSUE TRACTION

(71) Applicant: BOSTON SCIENTIFIC MEDICAL DEVICE LIMITED, Galway (IE)

(72) Inventors: Shoeb Khan, Gurgaon (IN); Anshul Chabra, Delhi (IN); Peeyush Tomar, Muzaffarnagar (IN); Amit Kumar, Noida (IN); Jahnavi Konduru, New Delhi (IN); Pallavi Singh, Gurgaon (IN); Ashutosh Maurya, Robertsganj (IN)

(73) Assignee: BOSTON SCIENTIFIC MEDICAL DEVICE LIMITED, Galway (IE)

(21) Appl. No.: 19/009,035

(22) Filed: Jan. 3, 2025

Related U.S. Application Data
(60) Provisional application No. 63/617,881, filed on Jan. 5, 2024.

(51) Int. Cl. A61B 17/02; A61B 17/00

(52) U.S. Cl. CPC A61B 17/0218; A61B 2017/00269

(57) ABSTRACT

A tissue traction device configured to apply traction to a target tissue within a patient's body by attaching an end of the tissue traction device to anchoring tissue spaced apart from the target tissue. The tissue traction device includes at least one weakened region facilitating severing of the tissue traction device therealong, as compared with surrounding non-weakened regions. The at least one weakened region may be visually identifiable to facilitate severing of the tissue traction device therealong within the patient. The tissue traction device may be formed of distinctly-formed sections, such as, without limitation, enclosed shapes with an aperture therethrough, such as loops.

### CROSS-REFERENCE TO RELATED APPLICATIONS

This application claims the benefit of priority under 35 U.S.C. § 119 to U.S. Provisional Application No. 63/617,881, filed Jan. 5, 2024, the entire disclosure of which is hereby incorporated by reference herein for all purposes.

### FIELD

The present disclosure relates to the field of tissue traction devices, systems, and methods, such as which may be used to retract tissue during an endoscopic resection or dissection procedure.

### BACKGROUND

Various surgical procedures involve lifting or separating target tissue (a designated section of tissue) at a treatment site, such as while the target tissue is still attached to the treatment site. A tissue traction element may be used to lift the target tissue away from the treatment site at which the procedure is being performed. In some instances, the target tissue is an unhealthy, diseased (i.e., cancerous, pre-cancerous etc.), or otherwise undesirable portion of tissue (that may be healthy or unhealthy). A “target tissue” may also include tissues that are suspected of being unhealthy or diseased, but which require surgical removal for verification of their disease status by biopsy. Endoscopic Submucosal Dissection (ESD) and Endoscopic Mucosal Resection (EMR) are examples of outpatient procedures for removing deep tumors from the gastrointestinal (GI) tract. Even though this technique can allow patients to recover faster and often with less pain than with open or laparoscopic surgical procedures, such techniques require a high degree of expertise, and therefore are not yet widely adopted. One of the largest time and complexity drivers is managing the tissue being dissected/resected. As the medical professional cuts the tissue, it becomes harder to visualize the cutting plane (tissue to be cut) as tissue remains above the cutting plane, and also because the medical professional needs to cut increasingly deeper into the tissue. Accurately and efficiently performing an endoscopic procedure, such as endoscopic tissue resection and/or dissection, includes the ability to maintain traction on the target tissue above the cutting plane as the boundaries of the target tissue are cut and the tissue under traction is being lifted. Various traction devices exist to apply traction to the target tissue (e.g., to lift the cut region of tissue away from surrounding tissue) to facilitate visualization and easy resection/dissection. Various traction devices include wire mechanisms, magnetic mechanisms, and reinforced polymeric elements. The device should have good flexibility as well as sufficient tensile strength to create traction and should not interfere with the tissue resection/dissection procedure with which it is used. However, known traction systems may present challenges with respect to maintaining and/or adjusting tension applied to the target tissue, possibly obstructing a medical professional's view of the target tissue and/or interfering with access and/or use of accessory tools at the target site. These complications may directly contribute to increased procedures time, complexity, and risk of perforation or bleeding. Accordingly, there remains a need for improved traction devices, systems, and methods.

### SUMMARY

This Summary is provided to introduce, in simplified form, a selection of concepts described in further detail below in the Detailed Description. This Summary is not intended to necessarily identify key features or essential features of the claimed subject matter, nor is it intended as an aid in determining the scope of the claimed subject matter. One of skill in the art will understand that each of the various aspects and features of the present disclosure may advantageously be used separately in some instances, or in combination with other aspects and features of the disclosure in other instances, whether or not described in this Summary. No limitation as to the scope of the claimed subject matter is intended by either the inclusion or non-inclusion of elements, components, or the like in this Summary.

In accordance with various principles of the present disclosure, a tissue traction device has a first end and a second end and further includes a first section configured and adapted for attachment to tissue at a first location within a patient's body; a second section configured and adapted for attachment to tissue at a second location within a patient's body spaced apart from the first location; and at least one weakened region between the first end and the second end configured and adapted to be severed by a cutting tool more readily than surrounding regions.

In some aspects, the first section and the second section are configured and adapted for attachment to tissue with a tissue-engagement member. In some aspects, the tissue traction device further includes a tissue-engagement member having a first jaw and a second jaw, wherein a first jaw is coupled with the first section. In some aspects, the first section comprises an attachment section sized to engage and to remain in place on a jaw of the tissue-engagement member.

In some aspects, the at least one weakened region is provided along at least one of the first section of the second section.

In some aspects, the at least one weakened region is provided between the first section and the second section.

In some aspects, the tissue traction device further includes a third section between the first section and the second section. In some aspects, the weakened region is positioned along an elongated section spacing apart the first section and the third section and/or the second section and the third section.

In some aspects, at least one of the first section or the second section comprises a loop.

In some aspects, the tissue traction device further includes an attachment section positioned within the first section and sized to engage and to remain in place on a jaw of a tissue-engagement member for attaching the tissue traction device to tissue at the first location.

In accordance with various principles of the present disclosure, an elongated tissue traction device, extending between a first end and a second end, includes two or more distinctly formed sections distinguishable from one another; and at least one weakened region along and/or between the two or more distinctly-formed sections and configured and adapted to be severed by a cutting tool more readily than surrounding regions.

In some aspects, the at least one weakened region is a stepped reduced-diameter portion along at least one of the two or more distinctly-formed sections.

In some aspects, the at least one weakened region is positioned along an elongated section spacing apart adjacent distinctly-formed sections.

In some aspects, the tissue traction device further includes an attachment section sized to engage and to remain in place on a jaw of a tissue-engagement member delivered with the tissue traction device to a target tissue site. In some aspects, the attachment section is positioned within a section at the first end of the tissue traction device.

In accordance with various principles of the present disclosure, a method of applying traction to tissue within a patient's body includes anchoring a first end of a tissue traction device to tissue at a first location within a patient's body; anchoring a second end of a tissue traction device to tissue at a second location within a patient's body spaced apart from the first location to apply traction to the tissue at the first location; severing the tissue traction device along a weakened region of the tissue traction device.

In some aspects, the method further includes severing the tissue traction device to release traction on the tissue at the first location. In some aspects, the method further includes removing the tissue at the first location.

In some aspects, the method further includes anchoring a portion of the tissue traction device between the first end and the second end to tissue at a third location within a patient's body.

In some aspects, the method further includes visually identifying the weakened region before severing the tissue traction device along the weakened region.

In one example of an embodiment, a traction device includes weakened regions along the perimeter of at least one of the two or more closed loop sections to facilitate severing of the traction device. The traction device optionally includes different sections formed as shapes distinguishable from one another. Optionally, the different sections are closed loops. It will be appreciated that a loop may be in any of a variety of shapes (e.g., need not be circular or oval or otherwise).

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to, e.g., fit within a working channel of a delivery catheter or endoscope. In the figures, identical or nearly identical or equivalent elements are typically represented by the same reference characters, and similar elements are typically designated with similar reference numbers differing in increments of 1000, with redundant description omitted. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1A illustrates an example of an environment in which devices, systems, and methods in accordance with various principles of the present disclosure may be used with an example of an embodiment of a traction device and system being implemented.

FIG. 1B illustrates an example of an environment as in FIG. 1A with the example of an embodiment of a traction device and system as in FIG. 1A being further implemented.

FIG. 1C illustrates a view of a detail of FIG. 1B showing a section of the traction device being severed.

FIG. 2 illustrates a plan view of an example of an embodiment of a traction device formed in accordance with various principles of the present disclosure.

FIG. 3 illustrates a plan view of an example of an embodiment of a traction device formed in accordance with various principles of the present disclosure.

FIG. 4 illustrates a plan view of an example of an embodiment of a traction device formed in accordance with various principles of the present disclosure.

FIG. 4A illustrates a cross-sectional view along line 4A-4A of FIG. 4.

FIG. 4B illustrates a cross-sectional view along line 4B-4B of FIG. 4.

FIG. 4C illustrates a cross-sectional view along line 4C-4C of FIG. 4.

FIG. 5 illustrates a plan view of an example of an embodiment of a traction device formed in accordance with various principles of the present disclosure.

FIG. 6 illustrates a plan view of an example of an embodiment of a traction device formed in accordance with various principles of the present disclosure.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and/or closest to a delivery device, and “distal” refers to the direction or location furthest from the user, such as when using the device (e.g., introducing the device into a patient, or during implantation, positioning, or delivery), and/or closest to a delivery device. “Longitudinal” means extending along the longer or larger dimension of an element. A “longitudinal axis” extends along the longitudinal extent of an element, though is not necessarily straight and does not necessarily maintain a fixed configuration if the element flexes or bends, and “axial” generally refers to along the longitudinal axis. However, it will be appreciated that reference to axial or longitudinal movement with respect to the above-described systems or elements thereof need not be strictly limited to axial and/or longitudinal movements along a longitudinal axis or central axis of the referenced elements. “Central” means at least generally bisecting a center point and/or generally equidistant from a periphery or boundary. As used herein, a “free end” of an element is a terminal end at which such element does not extend beyond. It will be appreciated that terms such as at or on or adjacent or along an end may be used interchangeably herein without intent to limit unless otherwise stated, and are intended to indicate a general relative spatial relation rather than a precisely limited location. Finally, reference to “at” a location or site is intended to include at and/or about the vicinity of (e.g., along, adjacent, proximate, etc.) such location or site. As understood herein, corresponding is intended to convey a relationship between components, parts, elements, etc., configured to interact with or to have another intended relationship with one another.

The present disclosure relates to a variety of devices, systems, and methods for applying traction to tissue within a body. A number of medical procedures, including, but not limited, those performed along the digestive and/or biliary tract, utilize medical devices to access tissue intended for excision (e.g., “target tissue”) within the body. For example, in some current medical procedures (e.g., endoscopic submucosal dissection (ESD), endoscopic mucosal resection (EMR), Peroral Endoscopic Myotomy (POEM), cholecystectomy, Video-Assisted Thoracoscopic Surgery (VATS)), medical professionals may utilize an endoscope or similar medical device during access and removal of diseased lesions. The endoscope may be capable of both accessing the target tissue site (the site at which the target tissue is located, including tissue surrounding the target tissue) while also permitting various tissue manipulating devices to be deployed therethrough. Such tissue manipulating devices include, without limitation, devices for resecting target tissue, which include, without limitation, cutting devices such as knives, scalpels, scissors, electrocauterization devices, end effectors, graspers, snares, forceps, dissectors, energy-based tissue coagulators or cutters, clamps, tissue staplers, tissue loops, clip appliers, suture delivering instruments, etc., the particular device not being critical to the present disclosure. It will be appreciated that terms such as medical tools, instruments, devices, etc., may be used interchangeably herein without intent to limit. Additionally, in some instances, an endoscope may incorporate features which assist the medical professional in visualizing and/or imaging the tissue dissection/resection procedure, such as to facilitate performance of the procedure. For example, some endoscopes may include a light and/or camera designed to illuminate and/or visualize the treatment site/target tissue area as the endoscope is navigated and positioned adjacent to the target tissue site. Additionally, some endoscopes may also include a lumen (e.g., a working channel) through which a further instrument, and optionally also a delivery sheath for the instrument, may be deployed and utilized. Additional visualization methods (e.g., fluoroscopy) may be alternatively or additionally employed. It will be appreciated that reference is made herein to tissue resection (and other grammatical forms thereof) for the sake of convenience, such term encompassing tissue dissection, cutting, manipulation, etc. (and other grammatical forms thereof) without intent to limit.

While physicians are becoming more proficient at resecting diseased lesions from within the body (e.g., within the digestive tract, abdominal cavity, thoracic cavity, etc.), present traction methods may continue to be inefficient to the physician. For example, in some instances poor visualization and poor ability to engage and manipulate tissue may result in a prolonged tissue dissection procedure. It may be desirable to lift or to retract the tissue out of the field of vision or out of the way of the instrument being used to perform the procedure. For instance, in some EMR/ESD procedures, physicians may use separate devices to provide a means of tissue traction, such as to move tissue with respect to surrounding tissue. Positioning and maneuvering (e.g., traction) of a resected tissue flap during and after resecting presents various challenges. Such procedures may include multiple device manipulations and/or exchanges, and accompanying extended procedure times. Such systems may be unable to maintain or adjust traction or tension applied to the target tissue, and/or may maintain or adjust traction or tension applied to the target tissue in an inefficient or inconsistent manner.

In accordance with various principles of the present disclosure, a tissue traction device is used to apply traction to tissue to facilitate performance of a procedure with respect to such tissue. In some aspects, the tissue traction device (which may be alternately referenced herein as a tether for the sake of convenience and without intent to limit) is elongated or otherwise shaped to facilitate extending from a target tissue site to a tissue anchoring site. More particularly, in some aspects, the tissue traction device has a first end configured to be engaged with target tissue at a target tissue site, and a second end configured to be engaged with anchoring tissue at a tissue anchoring site, with an elongated extent therebetween. It will be appreciated that terms such as engage (and other grammatical forms thereof) may be used interchangeably herein with terms such as, without limitation, couple, grasp, hold, clasp, clip, anchor, attach, affix, secure, etc. (and other grammatical forms thereof), without intent to limit. It will be appreciated that terms such as “anchoring tissue” and “tissue anchoring site” are used for the sake of convenience, and may alternately be referenced by terms such as traction tissue sites, anchoring sites, etc., to describe a location at which a portion or section or segment of a tissue traction may be engaged to apply traction to a target tissue site. The target tissue site is typically the site at which a portion or section or segment of the tissue traction device is initially engaged, such as to lift or otherwise to apply traction thereto. It will be appreciated that terms such as portion, section, segment, etc., may be used interchangeably herein without intent to limit unless otherwise specified. The tissue anchoring site typically is spaced apart from the target tissue site, such as across/on an opposite side of a body lumen from the target tissue. As such, engagement of the second end with anchoring tissue after engagement of the first end with target tissue allows for a traction force to be applied to the target tissue, such as to lift the target tissue away from the surrounding tissue. The traction force may be in the range of at least about 0.7 N to as much as about 4.7 N (including increments of 0.1 N therebetween). In some aspects, a tissue traction device formed in accordance with various principles of the present disclosure is stretchable and/or elongatable. It will be appreciated that the tether may advantageously be elastic and/or elastomeric (e.g., formed of rubber), though elasticity and/or stretchability are not necessarily aspects of a traction device formed in accordance with various principles of the present disclosure.

In some aspects, one or more anchoring-tissue-engaging sections are provided between the first end and the second end of the tissue traction device. It will be appreciated that terms such as portion, area, section, segment, etc., may be used interchangeably herein without intent to limit, reference generally being made to a section as a general region, and a segment as a particular part, for the sake of convenient differentiation and without intent to limit. Engagement of one or more additional engagement sections of the tissue traction device with further anchoring tissue spaced from the anchoring tissue to which the second end of the tissue traction device is engaged allows for the modification of magnitude and/or direction of the force vector applied to the target tissue by the tissue traction device. In some aspects, the one or more anchoring-tissue-engaging sections are configured and positioned to be engaged with tissue at the target tissue site directly or indirectly, such as with the use of tissue-engagement member. As the target tissue, to which the first end of the tissue traction device is engaged, is moved with respect to the surrounding tissue (e.g., cut with respect to surrounding tissue), the traction applied thereto by the tissue traction device may decrease. In accordance with various principles of the present disclosure, rather than moving the second end of the tissue traction device to another location to increase tension on the target tissue, one or more of the anchoring-tissue-engaging sections of the tissue traction device may be engaged with anchoring tissue to increase the traction applied to the target tissue. It will be appreciated that the additional anchoring-tissue-engaging sections of the tissue traction device may be engaged at essentially the same anchoring tissue site (depending on the size, shape, configuration, and/or location of the various traction-tissue-engagement sections) or at different anchoring tissue sites (such as increasingly further from the target tissue). For instance, if the distance between the target tissue and the various anchoring-tissue-engaging sections of the tissue traction device are different, different traction force vectors may be applied by the different anchoring-tissue-engaging segments even if engaged at a common traction tissue site.

In some aspects, a tissue traction device formed in accordance with various principles of the present disclosure is shaped to define different points of attachment of the tissue traction device to tissue, such as with the use of a tissue-engagement member. For instance, in some embodiments, the one or more anchoring-tissue-engaging segments/sections are formed as distinct grasping sections or segments, such as having borders and/or shapes and/or cross-sections distinct from another grasping section or segment. It will be appreciated that terms such as section or segment or area or portion or the like may be used interchangeably herein without intent to limit. The different anchoring-tissue-engaging sections of the tissue traction device may be distinguished from one another into different grasping segments defined along a section of the tissue traction device such as by a difference in shape or orientation with respect to another grasping segment of the tissue traction device (such as, without limitation, adjacent or adjoining the grasping segments). In some aspects, the anchoring-tissue-engaging sections are formed as closed shapes with an opening therethrough, such as to allow a portion of a tissue-engaging member (e.g., one of a pair of jaws of a tissue-engaging member) to be extended therethrough. For instance, one or more of the anchoring-tissue-engaging sections may be in the form of loops.

To facilitate release of traction (and/or retrieval of the target tissue and/or portions of the tissue traction device), a tissue traction device formed in accordance with various principles of the present disclosure includes weakened regions along the length thereof. It will be appreciated that the weakened regions are not so weak as to break under traction forces anticipated by a procedure with which the tissue traction device is designed to be used. Instead, the weakened regions are weakened to allow disruption thereof, such as by a cutting device. Any of a variety of cutting devices, such as knives, forceps, electrocautery knifes, snares, etc., may be used to apply sufficient force to a selected weakened region to disrupt (e.g., sever, break, cut, disrupt, etc., such terms being used interchangeably herein without intent to limit) the tissue traction device to release traction and/or to allow retrieval of the target tissue. Optionally, the cutting device is a device for resecting target tissue. The weakened regions may be formed along any portion of the tissue traction device, such as along an anchoring-tissue-engaging sections and/or between adjacent anchoring-tissue-engaging sections. As used herein, the term “weakened region” means an area of material that promotes or enhances severing of a structure. As such, a weakened region can be made by reducing a cross-sectional dimension of the tissue traction device at such region. The weakened region may have the same or different cross-sectional shape as surrounding regions of the tissue traction device which are not considered to be “weakened”, the present disclosure not being limited in this regard. Alternatively or additionally, the material may be altered to facilitate intentional cutting therethrough, without affecting the tensile strength (and thus not affecting the traction forces the tissue traction device may apply to target tissue). Alternatively or additionally, a mechanical or chemical modification to the tissue traction device may be made to the tissue traction device at the weakened region to create or enhance a weakened region. For instance, including, but not limited to, embossing, scoring, or cutting; or non-mechanical means, including, but not limited to, chemical etching; lasers, heat; or combinations of mechanical and non-mechanical means. It will be appreciated that, in some aspects, a portion of a tissue traction device, anchored by a tissue engagement member to anchoring tissue, may be left engaging the anchoring tissue. Such tissue traction device portion and tissue-engagement member will eventually naturally slough off.

Various embodiments of tissue traction devices, systems, and methods associated therewith will now be described with reference to examples illustrated in the accompanying drawings. Reference in this specification to “one embodiment,” “an embodiment,” “some embodiments”, “other embodiments”, etc. indicates that one or more particular features, structures, concepts, and/or characteristics in accordance with principles of the present disclosure may be included in connection with the embodiment. However, such references do not necessarily mean that all embodiments include the particular features, structures, concepts, and/or characteristics, or that an embodiment includes all features, structures, concepts, and/or characteristics. Some embodiments may include one or more such features, structures, concepts, and/or characteristics, in various combinations thereof. It should be understood that one or more of the features, structures, concepts, and/or characteristics described with reference to one embodiment can be combined with one or more of the features, structures, concepts, and/or characteristics of any of the other embodiments provided herein. That is, any of the features, structures, concepts, and/or characteristics described herein can be mixed and matched to create hybrid embodiments, and such hybrid embodiment are within the scope of the present disclosure. Moreover, references to “one embodiment,” “an embodiment,” “some embodiments”, “other embodiments”, etc. in various places in the specification are not necessarily all referring to the same embodiment, nor are separate or alternative embodiments necessarily mutually exclusive of other embodiments. It should further be understood that various features, structures, concepts, and/or characteristics of disclosed embodiments are independent of and separate from one another, and may be used or present individually or in various combinations with one another to create alternative embodiments which are considered part of the present disclosure. Therefore, the present disclosure is not limited to only the embodiments specifically described herein, as it would be too cumbersome to describe all of the numerous possible combinations and subcombinations of features, structures, concepts, and/or characteristics, and the examples of embodiments disclosed herein are not intended as limiting the broader aspects of the present disclosure. It should be appreciated that various dimensions provided herein are examples and one of ordinary skill in the art can readily determine the standard deviations and appropriate ranges of acceptable variations therefrom which are covered by the present disclosure and any claims associated therewith. The following description is of illustrative examples of embodiments only, and is not intended as limiting the broader aspects of the present disclosure.

It will be appreciated that common features are identified by common reference elements and, for the sake of brevity and convenience, and without intent to limit, the descriptions of the common features are generally not repeated. For purposes of clarity, not all components having the same reference number are numbered. It will be appreciated that, in the following description, elements or components similar among the various illustrated embodiments with reference numbers greater than 1000 are generally designated with the same reference numbers increased by a multiple of 1000 and redundant description is generally omitted for the sake of brevity. Moreover, certain features in one embodiment may be used across different embodiments and are not necessarily individually labeled when appearing in different embodiments. Accordingly, the common features are identified by common reference elements and, for the sake of brevity, the descriptions of the common features will not be repeated. For purposes of clarity, not all components having the same reference number are numbered, and individual elements among similar may be identified by a common reference number with a different reference letter at the end, the elements in general being indicated by simply the common reference number.

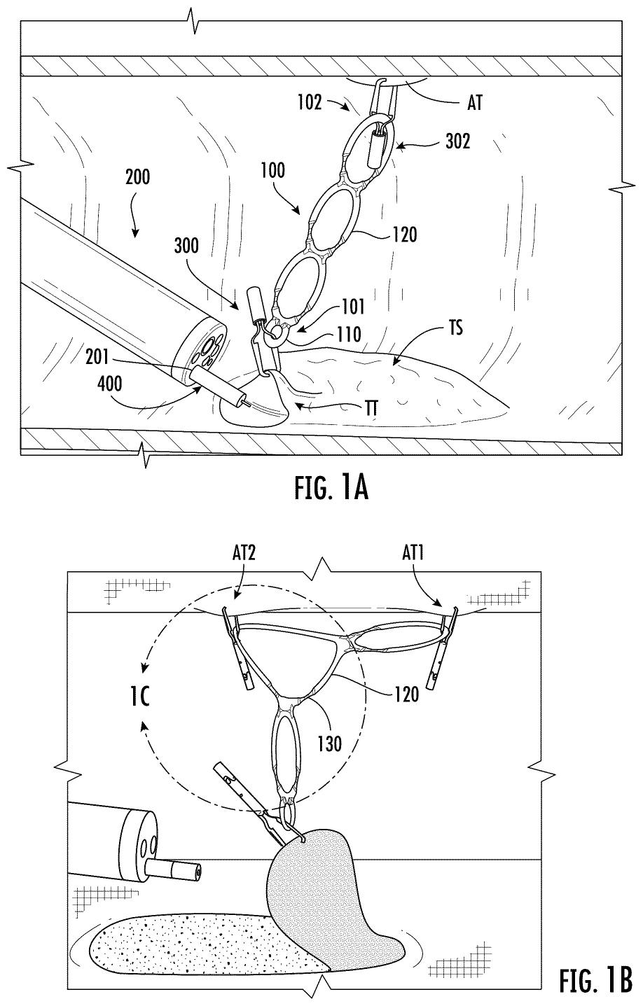

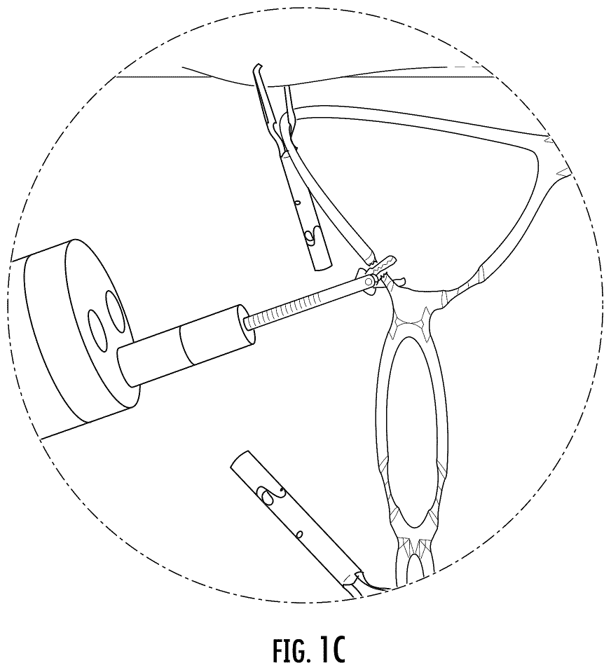

Turning now to the drawings, FIG. 1A and FIG. 1B illustrate examples of use of an example of an embodiment of a tissue traction device 100 in accordance with various principles of the present disclosure. The illustrated example of an embodiment of a tissue traction device 100 has a target-tissue-engaging first end 101 engaged (directly or indirectly, such as with the use of a tissue-engagement member 300 described in further detail below) with target tissue TT at a target tissue site TS, and an anchoring-tissue-engaging end 102 engaged (directly or indirectly, such as with the use of a second tissue-engagement member 302) with anchoring tissue AT spaced apart from the target tissue TT. Engaged as such, the tissue traction device 100 applies traction to the target tissue TT. It will be appreciated that reference to a first end or a second end of the tissue traction device is for the sake of simplicity, and is not to be understood as being limited to an end of an elongated element, but may also apply to other locations along an element, such as, without limitation, an elongated element.

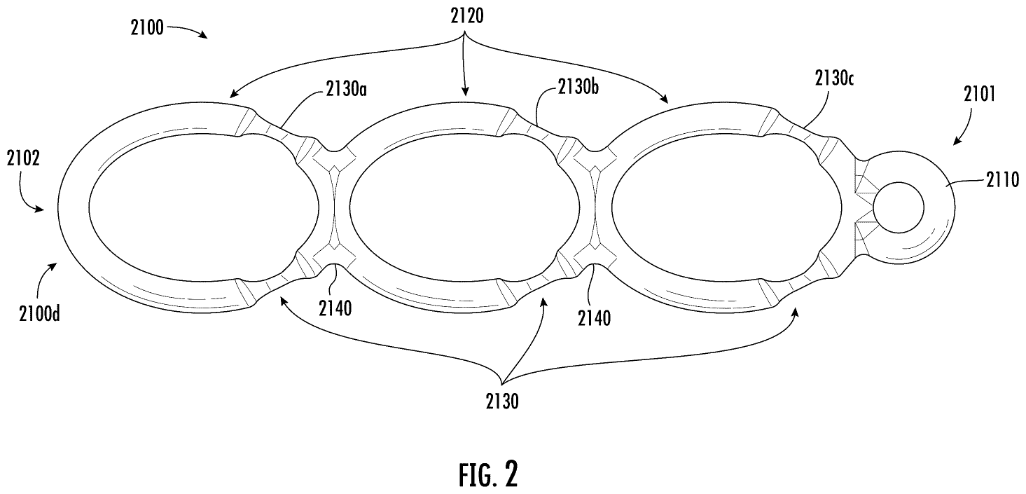

It will be appreciated that the tissue traction device 100 may be delivered to the target tissue site TS with any suitable delivery device 200. For instance, the tissue traction device 100 may be delivered within a tubular elongate member. In some aspects, the tubular elongate member is sufficiently flexible to navigate through tortuous body passages. It will be appreciated that reference to a body passage includes naturally existing passages (e.g., the colon) as well as medically created passages (e.g., a passage created with the use of a medical instrument, and not existing without medical intervention) or otherwise. In some aspects, the delivery device 200 is a medical scope, such as an endoscope, with a working channel 201 defined through the delivery device 200 and through which the tissue traction device 100 may be delivered. The tissue traction device 100 may be delivered in a generally extended configuration, or in a more compact configuration, the present disclosure not being limited in this regard. Preferably, the dimensions of the tissue traction device 100 are selected to fit within a range of inner diameters of a range of delivery devices 200 when the tissue traction device 100 is in any of a variety of delivery configurations within the delivery device 200. In some aspects, the tissue traction device 100 is delivered with a tissue-engagement member 300 engaging the first end 101 of the tissue traction device 100, as discussed in further detail below. In some aspects, the tissue traction device 100 includes an attachment section 110 to facilitate attachment of a tissue-engagement member 300 to the tissue traction device 100. In the example of an embodiment illustrated in FIG. 2, the attachment section 110 is a loop or an aperture formed within the first end 101 of the tissue traction device 100. In embodiments in which the attachment section 110 is at the first end 101 of the tissue traction device 100, the attachment section 110 may also be referenced as a primary loop 110. The attachment section 110 may be configured and adapted and sized to engage and to remain in place on a tissue-engagement member 300, such as by an interference fit. For instance, the attachment section 110 may have sufficient resiliency or elasticity to be stretched over a portion of a tissue-engagement member 300 (e.g., a jaw of a pair of jaws of a tissue-engagement member 300 in the form of a clip) and to retract over such portion of the tissue-engagement member 300 to be held in place such as by hoop strength or friction or otherwise. It will be appreciated that other configurations of an attachment section 110 which allow for secure attachment of a tissue-engagement member 300 thereto are within the scope of the present disclosure, the present disclosure not being limited to loops.

Once the tissue traction device 100 has been delivered to the target tissue site TS, the tissue-engagement member 300 may be engaged with tissue at the target tissue site TS, as illustrated in FIG. 1A. In particular, the tissue-engagement member 300 may be engaged with target tissue TT intended to be excised from surrounding tissue at the target tissue site TS to couple the first end 101 of the tissue traction device 100 with the target tissue TT so that the tissue traction device 100 may apply traction to the target tissue TT. More particularly, once the first end 101 of the tissue traction device 100 is coupled with the target tissue TT, the second end 102 of the tissue traction device 100 may be extended to an anchoring tissue site AT spaced apart from the target tissue site TS. In the schematically anatomical environment illustrated in FIG. 1A and FIG. 1B, the target tissue site TS is within a body lumen (e.g., small intestine, large intestine, esophagus, etc.) on one side of the body lumen, and the anchoring tissue site AT is on a side generally opposite the side of the body lumen at which the target tissue site TS is located, such as to optimize traction to be applied by the tissue traction device 100. In other environments, such as a body cavity (e.g., within an organ such as a stomach), the anchoring tissue site AT is selected to be spaced far enough from the target tissue site TS such that the tissue traction device 100 may apply the appropriate/desired traction to the target tissue TT. It will be appreciated that one of ordinary skill in the art will recognize that selection of an anchoring tissue site AT may include considerations in addition to location and distance from the target tissue site TS, such as the length, elasticity, stretchability, maximum elongation, and/or other material properties of the tissue traction device 100.

As illustrated in FIG. 1A, the second end 102 of the tissue traction device 100 may be anchored with respect to the anchoring tissue site AT with the assistance of a second tissue-engagement member 302. The second tissue-engagement member 302 may be similar in configuration to the first tissue-engagement member 300 used to anchor the first end 101 of the tissue traction device 100 with the target tissue TT, or may be a different type or configuration of tissue-engagement member, the present disclosure not being limited in this regard. In some aspects, the second end 102 includes an anchoring-tissue-engaging section 120 configured and adapted to facilitate grasping thereof and attachment to tissue for anchoring thereto. In some aspects, the anchoring-tissue-engaging section 120 is a closed shape with an opening therethrough configured and adapted for at least a portion of a tissue-engagement member 300, 302 (e.g., a jaw of a pair of jaws of a tissue-engagement member 300, 302) to extend therethrough. In some aspects, the tissue traction device 100 includes more than one anchoring-tissue-engaging section 120, as will be described in further detail below.

With a tissue traction device 100 engaged with target tissue TT along the target tissue site TS and also engaged with tissue at an anchoring tissue site AT, the medical professional may commence cutting the target tissue TT, such as to excise at least a portion of the target tissue TT. A cutting instrument 400 may be advanced through the delivery device 200 (e.g., through a working channel 201 defined through the delivery device 200, such as through which the tissue traction device 100 is delivered, or an additional working channel 201) and to the target tissue site TS. As the cutting instrument 400 commences cutting tissue at the target tissue site TS, the target tissue TT begins to separate (e.g., to lift) from surrounding tissue at the target tissue site TS. The traction applied to the target tissue TT by the tissue traction device 100 may thereby be affected (e.g., reduced). In order to modify (e.g., increase the magnitude and/or alter the direction of) the force vector of the traction applied by the tissue traction device 100, one or more anchoring-tissue-engaging section 120 of the tissue traction device 100, between the first end 101 and the second end 102 of the tissue traction device 100, may be anchored with respect to a second anchoring tissue site AT2, with the second end 102 still anchored with respect to the first anchoring tissue site AT1, as illustrated in FIG. 1B. The one or more anchoring-tissue-engaging sections 120 are configured and adapted for attachment to tissue, such as anchoring tissue spaced apart from the target tissue site TS. In accordance with various principles of the present disclosure, one or more anchoring-tissue-engaging sections 120 are provided between the first end 101 and the second end 102 of the tissue traction device 100. Further in accordance with various principles of the present disclosure, the anchoring-tissue-engaging sections 120 may be sized, shaped, configured, and/or dimensioned differently from one another so that the anchoring-tissue-engaging sections 120 are distinct and/or distinguishable from one another and/or may be grasped and anchored to an anchoring tissue site AT individually to impart different force vectors on the target tissue TT. In the examples of embodiments illustrated in the accompanying figures, each of the anchoring-tissue-engaging sections 120 is an enclosed shape with an aperture/opening therethrough to facilitate grasping thereof by a tissue-engagement member 300 to anchor with respect to an anchoring tissue site AT. The location at which the tissue-engagement member 300 engages the anchoring-tissue-engaging section 120 need not be limited, and instead may be at any of a variety of points around the perimeter of the anchoring-tissue-engaging section 120. It will be appreciated that other shapes or configurations of anchoring-tissue-engaging sections 120 are within the scope of the present disclosure, the present disclosure not being limited by the examples of embodiments illustrated in the accompanying figures.

Once cutting and/or any additional procedure has been performed at or with respect to the target tissue TT and/or the target tissue site TS, it may be desirable to remove at least a portion of the tissue traction device 100 from the patient's body. In accordance with various principles of the present disclosure, the tissue traction device 100 is configured to facilitate detachment by including one or more weakened regions 130. The weakened regions 130 are configured and adapted to facilitate severing of the tissue traction device 100 therealong. For instance, in some embodiments, the weakened region 130 has a smaller cross-sectional dimension than other (e.g., surrounding) regions of the tissue traction device 100. The smaller cross-sectional dimension, and/or the longitudinal/perimetral extent of the weakened regions 130 is selected so as not to adversely affect (e.g., reduce) the tensile strength of the tissue traction device 100 in such region, so as not to affect the ability of the tissue traction device 100 to apply a traction force to the target tissue TT. However, the reduced cross-sectional dimension is sufficiently reduced such that the weakened regions 130 is identifiable within the patient's body (e.g., with the use of an imaging system, such as a visualization device of a delivery device 200 such as a medical scope) and easier to sever than other regions of the tissue traction device 100. In some aspects, the tissue traction device 100 may be cut with the use of the same cutting instrument 400 used to cut tissue at the target tissue site TS, such as illustrated in FIG. 1C. In some aspects, a portion of the tissue traction device 100 grasped by a tissue-engagement member 300 may remain coupled with tissue within the patient. However other portions of the tissue traction device 100, such as a portion attached to target tissue TT which is excised and removed from the patient, may be removed, such as upon severing the tissue traction device 100 along a weakened region 130 thereof. It will be appreciated that portions of the tissue traction device 100 which remain attached to tissue within the patient (along with any tissue-engagement member 300 coupled therewith) ultimately are expected to slough off during natural functioning of the tissue.

Weakened regions 130 formed in accordance with various principles of the present disclosure may be provided at one or more points along the tissue traction device 100, such as along the first end 101, the second end 102, an anchoring-tissue-engaging section 120, and/or between adjacent anchoring-tissue-engaging sections 120. The weakened regions 130 typically extend along at least approximately 15% of the perimeter of an anchoring-tissue-engaging section 120 (e.g., to facilitate identification thereof for severing while within the patient's body), up to approximately 20% of the perimeter. However, in some embodiments, the weakened regions 130 may extend up to approximately 80% of the perimeter (including increments of 1% from 15% to 80%) of the anchoring-tissue-engaging section 120. The perimetral extent of the weakened regions 130 may allow for use of a variety of different tissue-engagement members 300 of various dimensions to grasp onto the non-weakened regions of a given anchoring-tissue-engaging section 120, yet to allow a cutting instrument 400 to access a weakened region 130. Various examples of embodiments of tissue traction devices with various configurations of weakened regions formed in accordance with various principles of the present disclosure are illustrated in FIGS. 2-6, as will now be described.

Turning to FIG. 2, the illustrated example of an embodiment of a tissue traction device 2100 has a first end 2101, a second end 2102, and one or more anchoring-tissue-engaging section 2120 extending therebetween. More particularly, an attachment section 2110 may be provided along the first end 2101, such as to facilitate attachment of a tissue-engagement member 300 (such as illustrated in FIG. 1) thereto, such as for delivery with the tissue traction device 2100, such as described above with respect to FIGS. 1A-1C. The second end 2102 of the tissue traction device 2100 illustrated in FIG. 2 is in the form of a loop, although other configurations are within the scope of the present disclosure. In some aspects, load-bearing bridge sections 2140 are formed between adjacent anchoring-tissue-engaging sections 2120, such as to increase the load bearing capability of the tissue traction device 2100 while applying traction to target tissue TT.

In accordance with various principles of the present disclosure, one or more weakened regions 2130 are formed along the longitudinal extent of the tissue traction device 2100, between the first end 2101 and the second end 2102 of the tissue traction device 2100. For instance, in some aspects, a weakened region 2130a may be provided along an anchoring-tissue-engaging section 2120 provided along the second end 2102 of the tissue traction device 2100. Such weakened region 2130a may allow separation/detachment of an increased extent of the tissue traction device 2100 from the anchoring tissue site AT at which the second end 2102 of the tissue traction device 2100 may remain attached via a tissue-engagement member 302 (such as illustrated in FIG. 1A or FIG. 1B). In some aspects, the weakened regions 2130a is proximally spaced apart from the distalmost end 2100d of the tissue traction device 2100, such as to facilitating severing the second end 2102 of the tissue traction device 2100 at a location at which the cutting instrument 400 has sufficient space as to not affect (e.g., contact) the anchoring tissue site AT.

As may be further appreciated with reference to the example of an embodiment of a tissue traction device 2100 illustrated in FIG. 2, two or more anchoring-tissue-engaging section 2120 may be provided between the first end 2101 and the second end 2102 of the tissue traction device 2100. One or both of the illustrated anchoring-tissue-engaging sections 2120 may include one or more weakened regions 2130 (e.g., additional weakened regions 2130b, 2130c) formed in accordance with various principles of the present disclosure. The weakened regions 2130 may be formed at any location along the perimeter or longitudinal extent of an anchoring-tissue-engaging section 2120. For instance, the weakened regions 2130 may be spaced apart along the extent of the tissue traction device 2100 between the first end 2101 and the second end 2102 thereof. Alternatively or additionally, the weakened regions 2130 may all be along the same location (e.g., closer to the first end 2101 or closer to the second end 2102 of the tissue traction device 2100) along a given anchoring-tissue-engaging section 2120 (such as all closer to the first end 2101 as illustrated in FIG. 2), or each weakened region 2130 may be along a different longitudinal/perimetral position along a given anchoring-tissue-engaging section 2120.

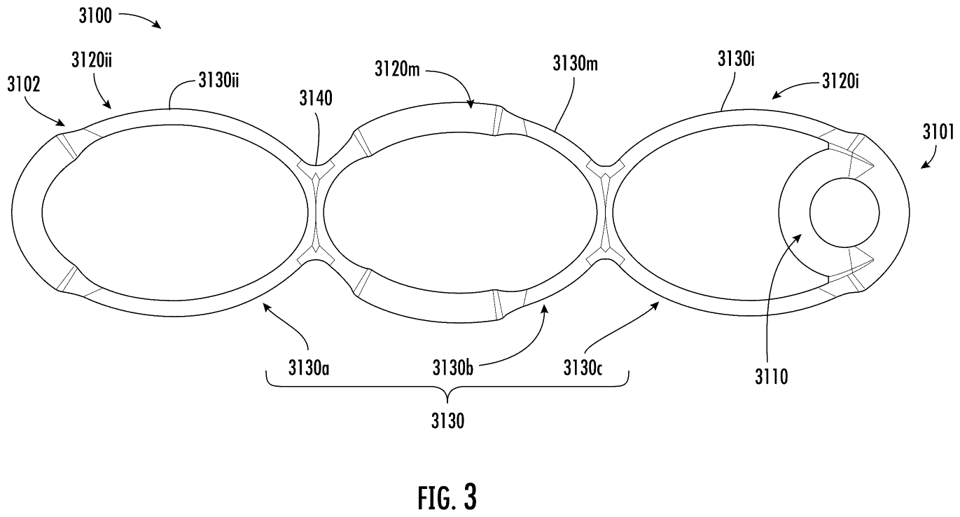

As may be appreciated with reference to the example of an embodiment of a tissue traction device 3100 illustrated in FIG. 3, the longitudinal/perimetral extent of the weakened regions 3130 of a tissue traction device formed in accordance with various principles of the present disclosure may vary from that shown in the example of an embodiment illustrated in FIG. 2. More particularly, the weakened regions 3130 of the tissue traction device 3100 illustrated in FIG. 3 may have longer longitudinal/perimetral extents than the weakened regions 2130 of the tissue traction device 2100 illustrated in FIG. 2. In some aspects, the weakened regions 3130 of the tissue traction device 3100 may not all have the same longitudinal/perimetral extent. More particularly, in the example of an embodiment of a tissue traction device 3100 illustrated in FIG. 3, the anchoring-tissue-engaging section 3120i adjacent the first end 3101 and the anchoring-tissue-engaging section 3120ii adjacent the second end 3102 of the tissue traction device 3100 may have weakened regions 3130i, 3130ii, respectively, which have longer longitudinal/perimetral extents than the weakened region 3130m of one or more intermediate anchoring-tissue-engaging sections 3120m. Various alternative configurations, such as the weakened regions 3130m of an intermediate anchoring-tissue-engaging section 3120m being longer than other weakened regions 3130 of the tissue traction device 3100 are also within the scope of the present disclosure. Alternatively or additionally, as noted above with reference to the tissue traction device 2100 illustrated in FIG. 2, the weakened regions 3130 of the tissue traction device 3100 illustrated in FIG. 3 may be provided at different locations along a given anchoring-tissue-engaging section 3120. For instance, one or more of the anchoring-tissue-engaging sections 3120 (e.g., adjacent the second end 3102 and/or an intermediate anchoring-tissue-engaging section 3120m) may include a weakened regions 3130 closer to the first end 3101 of the tissue traction device 3100, whereas one or more anchoring-tissue-engaging sections 3120 (e.g., an anchoring-tissue-engaging section 3120i closest to the first end 3101) may include a weakened regions 3130 closer to the second end 3102 of the tissue traction device 3100. Other combinations/positions/extents of weakened regions 3130 are within the scope of the present disclosure, the present disclosure not being limited by the example of an embodiment illustrated in FIG. 3.

It will be appreciated that the tissue traction device 3100 also includes an alternate position for an attachment section 3110 than illustrated in FIG. 1 and FIG. 2. More particularly, instead of extending away from an adjacent anchoring-tissue-engaging section 3120, the attachment section 3110 of the tissue traction device 3100 illustrated in FIG. 3 is positioned within the boundaries of the anchoring-tissue-engaging section 3120i closest to the first end 3101 of the tissue traction device 3100 (e.g., within the open space defined within the anchoring-tissue-engaging section 3120i). Such formation of an attachment section 3110 may allow for a thicker/larger cross-sectional area of the attachment section 3110 to counter the smaller overall dimension of the attachment section 3110 (such as compared to the anchoring-tissue-engaging sections 3120) and/or to provide additional holding strength (e.g., hoop strength) to the attachment section 3110 to securely be mounted and attached to a portion of a tissue-engagement member 300. Other configurations are within the scope of the present disclosure, the present disclosure not being limited to the longitudinal/perimetral extents illustrated in FIG. 3 or FIG. 2. In some aspects, like the tissue traction device 2100 of FIG. 2, the tissue traction device 3100 of FIG. 3 may include load-bearing bridge sections 3140 between adjacent anchoring-tissue-engaging sections 3120, such as to increase the load bearing capability of the tissue traction device 3100 while applying traction to target tissue TT.

The weakened regions 2130, 3130 of the tissue traction devices 2100, 3100 illustrated in FIG. 2 and FIG. 3, respectively, are generally configured and dimensioned to be distinct/distinguishable from adjacent regions of the tissue traction devices 2100, 3100. For instance, the weakened regions 2130, 3130 may be considered by at least medical professionals to be readily visually identifiable within the body, such as with conventional visualization means (e.g., an optical system of an endoscope). In some aspects, the weakened regions 2130, 3130 may have a generally stepped reduction in cross-sectional area and/or diameter relative to surrounding regions of the tissue traction devices 2100, 3100 illustrated in FIG. 2 and FIG. 3. It will be appreciated that “stepped reduction”, as used herein, is not limited to a sharp reduction (e.g., such as a square wave), but may include a short taper between the weakened regions 2130, 3130 sufficient to minimize sharp ends, but not so long in its perimetral/longitudinal extent as to render the transition to appear more as a taper than as a step. One of ordinary skill in the art may appreciate appropriate configurations in accordance with various principles of the present disclosure with reference to FIG. 2 and FIG. 3 as examples.

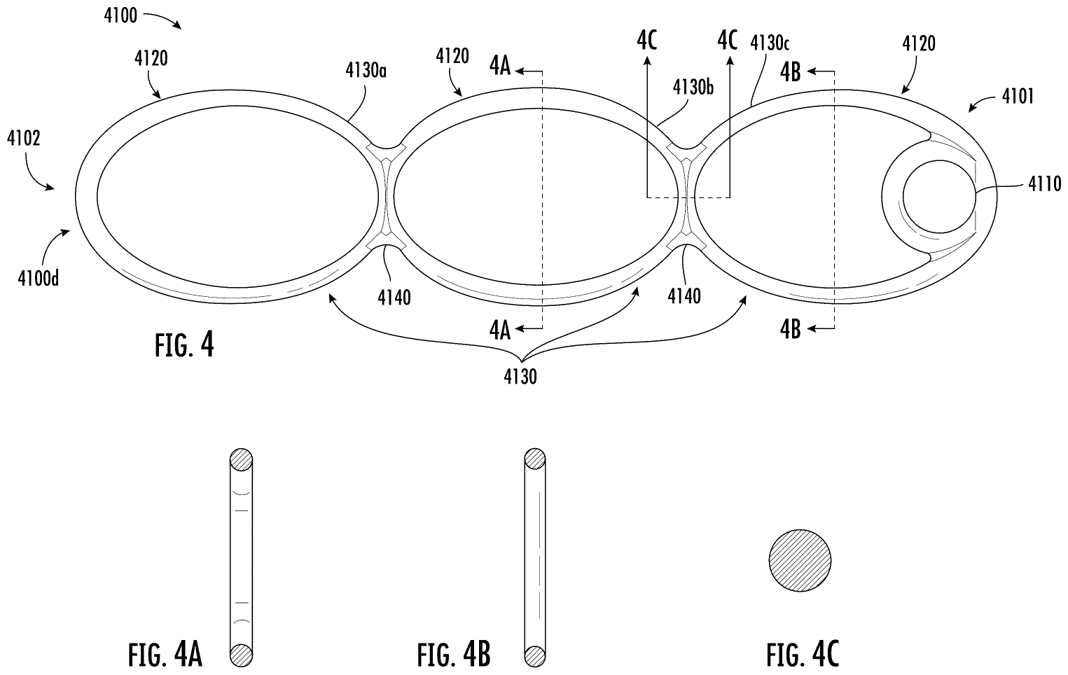

It will be appreciated that, in some aspects, the weakened regions of a tissue traction device formed in accordance with various principles of the present disclosure may not be as distinct from other sections of the tissue traction device. Instead, the reduced cross-sectional dimension of weakened regions 4130 of a tissue traction device 4100 formed in accordance with various principles of the present disclosure may be a less distinguishable reduction, such as illustrated in FIG. 4, FIG. 4A, FIG. 4B, and FIG. 4C. More particularly, the tissue traction device 4100 illustrated in FIG. 4 have anchoring-tissue-engaging sections 4120 has weakened regions 4130 which have gradual transitions (e.g., not well-defined stepped decreases in thickness, such as in the examples of embodiments illustrated in FIG. 2 and FIG. 3) to the weakened regions 4130 thereof. As may be appreciated upon comparison of FIG. 4A and FIG. 4B, the cross-sectional area of a non-weakened section of an anchoring-tissue-engaging section 4120, such as along line 4A-4A of FIG. 4 (as illustrated in FIG. 4A), is larger than the cross-sectional area of at least a portion of a weakened region 4130 of the anchoring-tissue-engaging section 4120, such as along line 4B-4B of FIG. 4 (as illustrated in FIG. 4B). In some aspects, like the tissue traction devices 2100, 3100 of FIG. 2, and FIG. 3, respectively, the tissue traction device 4100 of FIG. 4 may include load-bearing bridge sections 4140 between adjacent anchoring-tissue-engaging sections 4120, such as to increase the load bearing capability of the tissue traction device 4100 while applying traction to target tissue TT. As may be appreciated with reference to FIG. 4C, showing a cross-section a bridge section 4140 along line 4C-4C of FIG. 4, the cross-sectional area of the bridge section 4140 is larger than the cross-sectional area of at least a portion of a weakened region 4130 of the anchoring-tissue-engaging section 4120 such as illustrated in FIG. 4B. Optionally, the cross-sectional area of the bridge section 4140 is larger than (or at least as large as) the cross-sectional area of the anchoring-tissue-engaging section 4120 illustrated in FIG. 4A.

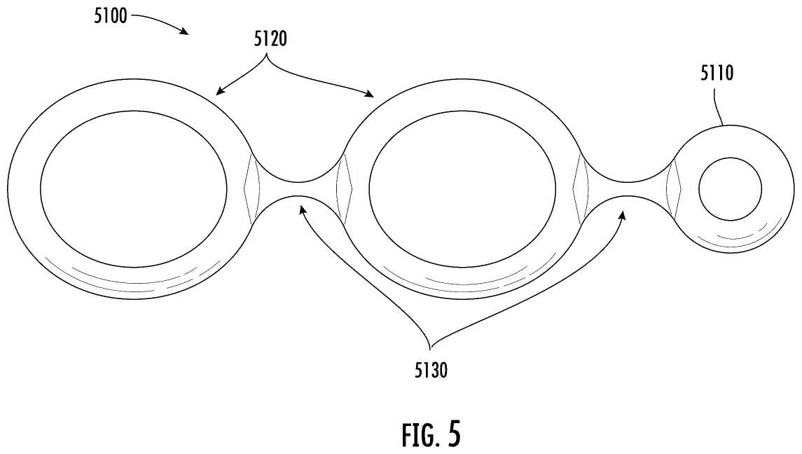

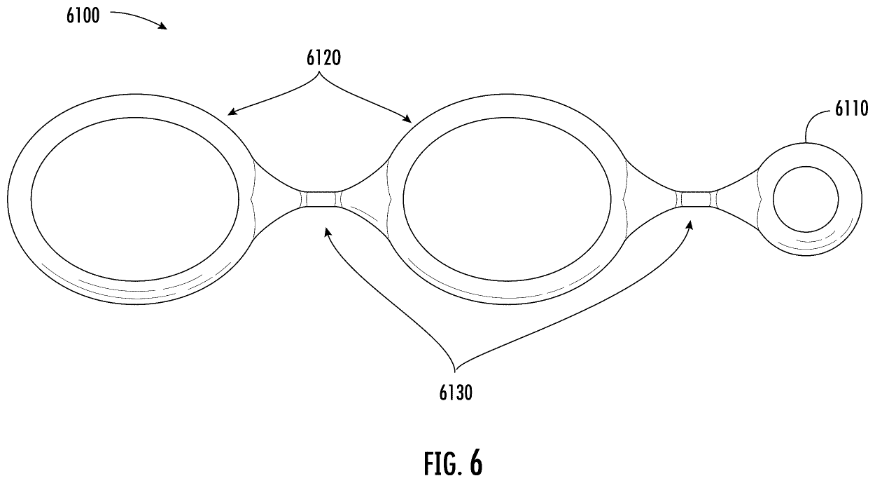

In accordance with various principles of the present disclosure, instead of being formed along an anchoring-tissue-engaging section of a tissue traction device, a weakened region may be formed between anchoring-tissue-engaging sections. For instance, in the examples of embodiments of tissue traction devices 5100 and 6100 illustrated, respectively, in FIG. 5 and FIG. 6, the weakened regions 5130, 6130 are formed between anchoring-tissue-engaging sections 5120, 6120, such as in the form of separate sections of different shape than proximal and distal anchoring-tissue-engaging sections 5120, 6120. In the examples of embodiments illustrated in FIG. 5 and FIG. 6, the weakened regions 5130, 6130 are elongated sections spacing apart adjacent anchoring-tissue-engaging section 5120, 6120. As may be appreciated upon comparison of the tissue traction device 5100 illustrated in FIG. 5 and the tissue traction device 6100 illustrated in FIG. 6, the relative sizes, shapes, configurations, and/or dimensions of the anchoring-tissue-engaging sections 5120, 6120 and the weakened regions 5130, 6130 may be varied without detracting from principles of the present disclosure. For example, the anchoring-tissue-engaging sections 5120 of the tissue traction device 5100 illustrated in FIG. 5 are more rounded than the more elliptical anchoring-tissue-engaging section 6120 of the tissue traction device 6100 illustrated in FIG. 6. Alternatively or additionally, the weakened regions 6130 of the tissue traction device 6100 may be more elongated, defining greater distances between adjacent anchoring-tissue-engaging section 6120 than the weakened regions 5130 of the tissue traction device 5100. It will be appreciated that the attachment sections 5110, 6110 of the tissue traction devices 5100, 6100, respectively, may be separated from the nearest anchoring-tissue-engaging section 5120, 6120 by a weakened region 5130, 6130, such as to facilitate separation of the portion of the tissue traction device 5100, 6100 attached to the target tissue TT being excised for separation and removal therewith.

It will be appreciated that a tissue traction device as described herein may be formed of any of a variety of biocompatible materials, such as polymers including, without limitation, polyethylene, polypropylene, nylon, etc., capable of exerting the desired traction forces yet also capable of being severed along a weakened region thereof. Such materials may be used to form the tissue traction device in any of a variety of manners, including, without limitation, injection molded, three-dimensional printing, vacuum casting, etc., known or heretofore known in the art. As may be appreciated, a variety of different shapes and sizes of tissue traction devices may be formed in accordance with various principles of the present disclosure. A typical tissue traction device formed in accordance with various principles of the present disclosure may have a thickness of at least about 0.3 mm to as great as about 0.8 mm (including increments of 0.1 mm therebetween), although the present disclosure need not be limited in this regard. A typical tissue traction device formed in accordance with various principles of the present disclosure may have a length of at least about 15 mm to as great as about 25 mm (including increments of 0.1 mm therebetween), although the present disclosure need not be limited in this regard. As noted above, the anchoring-tissue-engaging sections may be closed shapes, typically with an opening therethrough. For instance, the anchoring-tissue-engaging sections may be circular, oval, elliptical, etc., or other shapes which preferably do not have sharp edges or other configurations which may interfere with operation thereof. The outer diameter of an anchoring-tissue-engaging section may be as small as about 3 mm to as large as about 6 mm (including increments of 0.1 mm therebetween), such as to allow a sufficient extent along which the anchoring-tissue-engaging section may be grasped or otherwise engaged by a tissue-engagement member while within a patient's body. The inner diameter of the aperture may be at least about 1.3 mm or at least about 1.6 mm (including increments of 0.1 mm therebetween) to allow sufficient room for at least a portion of a tissue-engagement member to extend therethrough. Provision of more than one anchoring-tissue-engaging section provides greater flexibility to the medical professional to reposition the tissue traction device during a given procedure. In some aspects, the cross-sectional shape of the tissue traction device is circular or oval, such as to facilitate manufacture thereof. In some embodiments, the tissue traction devices may be color coded, such as to indicate size, dimension, strength, intended anatomical use, etc.

It will be appreciated that any of a variety of tissue-engagement members 300 may be used with a tissue traction device of the present disclosure. In some aspects, as described above, a portion of the tissue-engagement member 300 is coupled with the tissue traction device to remain connected with the tissue traction device during delivery and deployment. The tissue-engagement member 300 may be engaged with, mounted on, coupled to, attached to, etc., a tissue traction device formed in accordance with various principles of the present disclosure either directly or indirectly. The tissue-engagement member may alternately be known or referenced as a tissue fastener or clip or other mechanical securing device (e.g., a hemostatic clip, clamp, grasper, basket, gripper, magnet, adhesive, etc.), without intent to limit. In some embodiments, the tissue-engagement member is repositionable after being partially deployed. For example, the tissue-engagement member may be configured to allow for the tissue-engagement member to be releasably engaged (e.g., closed, but not locked, into engagement) with tissue when in a first configuration, and locked against opening out of engagement with tissue when in a second closed configuration. In some embodiments, the tissue-engagement member has grasper arms or jaws selectively movable away from each other to engage tissue therebetween, and movable towards each other to grasp the engaged tissue. The grasper arms may be hinged together (e.g., as a single piece), or separately formed and movable with respect to each other, such as by being pivotable about a pivot point. The grasper arms may have one or more additional grasping features, such as a sawtooth or crenulated profile or teeth, at ends and/or along edges of the grasper arms. In some embodiments, the tissue-engagement member is movable with respect to the tissue traction device, even when coupled thereto. Movement, such as rotation (e.g., 360° rotation), of the tissue-engagement member may be controlled by a proximal control knob, dongle, or other actuator element. In some embodiments, the tissue-engagement member may be maneuvered, e.g., rotated, with one-to-one correspondence between movement of a control knob and the tissue-engagement member.

It will also be appreciated that a delivery device used herewith may be any suitable size, cross-sectional shape or area, and/or configuration permitting introduction and passage of not only a tissue traction device formed in accordance with various principles of the present disclosure, optionally with a tissue-engagement member coupled therewith, but also one or more additional medical instruments. It is generally beneficial for the delivery device to be steerable, and the delivery device may have different areas of different flexibility or stiffness to promote steerability. The delivery device may be in the form of a flexible elongate tubular member and may include one or more working channels or lumens extending substantially longitudinally (axially) between the proximal end and the distal end of the delivery device.

It is to be understood by one of ordinary skill in the art that the present discussion is a description of illustrative examples of embodiments only, and is not intended as limiting the broader aspects of the present disclosure. Various further benefits of the various aspects, features, components, and structures of tissue traction devices such as described above, in addition to those discussed above, may be appreciated by those of ordinary skill in the art. It will be appreciated that all apparatuses and methods discussed herein are examples of apparatuses and/or methods implemented in accordance with one or more principles of this disclosure. These examples are not the only way to implement these principles but are merely examples, not intended as limiting the broader aspects of the present disclosure. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. It should be apparent to those of ordinary skill in the art that variations can be applied to the disclosed devices, systems, and/or methods, and/or to the sequence of steps of the method described herein without departing from the concept, spirit, and scope of the disclosure. It will be appreciated that various features described with respect to one embodiment typically may be applied to another embodiment, whether or not explicitly indicated. The various features hereinafter described may be used singly or in any combination thereof. Therefore, the present invention is not limited to only the embodiments specifically described herein, and all substitutes and modifications apparent to those skilled in the art are deemed to be within the spirit, scope, and concept of the disclosure as defined by the appended claims.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description. Tubular elongate member 1200 tubular elongate member 1200

[symbol]

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. As used in this specification and the appended claims, the term “or” is generally employed in its sense including “and/or” unless the content clearly dictates otherwise. As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, engaged, joined, etc.) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the terms “comprises”, “comprising”, “includes”, and “including” do not exclude the presence of other elements, components, features, groups, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. A tissue traction device having a first end and a second end and comprising:
a first section configured and adapted for attachment to tissue at a first location within a patient's body;
a second section configured and adapted for attachment to tissue at a second location within a patient's body spaced apart from the first location; and
at least one weakened region between the first end and the second end configured and adapted to be severed by a cutting tool more readily than surrounding regions.

2. The tissue traction device of claim 1, wherein said first section and said second section are configured and adapted for attachment to tissue with a tissue-engagement member.

3. The tissue traction device of claim 2, further comprising a tissue-engagement member having a first jaw and a second jaw, wherein a first jaw is coupled with said first section.

4. The tissue traction device of claim 3, wherein said first section comprises an attachment section sized to engage and to remain in place on a jaw of said tissue-engagement member.

5. The tissue traction device of claim 1, wherein said at least one weakened region is provided along at least one of said first section of said second section.

6. The tissue traction device of claim 1, wherein said at least one weakened region is provided between said first section and said second section.

7. The tissue traction device of claim 1, further comprising a third section between said first section and said second section.

8. The tissue traction device of claim 7, wherein said weakened region is positioned along an elongated section spacing apart said first section and said third section and/or said second section and said third section.

9. The tissue traction device of claim 1, wherein at least one of said first section or said second section comprises a loop.

10. The tissue traction device of claim 1, further comprising an attachment section positioned within said first section and sized to engage and to remain in place on a jaw of a tissue-engagement member for attaching said tissue traction device to tissue at the first location.

11. An elongated tissue traction device extending between a first end and a second end, said elongated tissue traction device comprising:
two or more distinctly formed sections distinguishable from one another; and
at least one weakened region along and/or between said two or more distinctly-formed sections and configured and adapted to be severed by a cutting tool more readily than surrounding regions.

12. The tissue traction device of claim 11, wherein said at least one weakened region is a stepped reduced-diameter portion along at least one of said two or more distinctly-formed sections.

13. The tissue traction device of claim 11, wherein said at least one weakened region is positioned along an elongated section spacing apart adjacent distinctly-formed sections.

14. The tissue traction device of claim 11, further comprising an attachment section sized to engage and to remain in place on a jaw of a tissue-engagement member delivered with said tissue traction device to a target tissue site.

15. The tissue traction device of claim 14, wherein said attachment section is positioned within a section at the first end of said tissue traction device.

16. A method of applying traction to tissue within a patient's body, said method comprising:
anchoring a first end of a tissue traction device to tissue at a first location within a patient's body;
anchoring a second end of a tissue traction device to tissue at a second location within a patient's body spaced apart from the first location to apply traction to the tissue at the first location;
severing the tissue traction device along a weakened region of the tissue traction device.

17. The method of claim 16, further comprising severing the tissue traction device to release traction on the tissue at the first location.

18. The method of claim 17, further comprising removing the tissue at the first location.

19. The method of claim 16, further comprising anchoring a portion of the tissue traction device between the first end and the second end to tissue at a third location within a patient's body.

20. The method of claim 16, further comprising visually identifying the weakened region before severing the tissue traction device along the weakened region.
